(19) United States
(12) Patent Application Publication
(10) Pub. No.: US 2015/0252317 A1
(43) Pub. Date: Sep. 10, 2015

(54) DISPOSABLE BIOREACTOR WITH FILTRATION SYSTEM

(71) Applicant: FloDesign Sonics, Inc., Wilbraham, MA (US)

(72) Inventors: Bart Lipkens, Hampden, MA (US); Louis Masi, Longmeadow, MA (US); Walter M. Presz, JR., Wilbraham, MA (US); Thomas J. Kennedy, III, Wilbraham, MA (US)

(21) Appl. No.: 14/644,036

(22) Filed: Mar. 10, 2015

Related U.S. Application Data
(60) Provisional application No. 61/950,309, filed on Mar. 10, 2014.

(51) Int. Cl. C12M 1/00

(52) U.S. Cl. CPC C12M 47/10; C12M 23/14

(57) ABSTRACT

A disposable bioreactor system includes a bag having a first end and a second end. An acoustophoresis device is disposed at the first end of the bag, and is separable from the bag. An actuating mechanism is operably connected to the second end of the bag. The actuating mechanism is configured to move the second end of the bag towards the acoustophoresis device, so that fluids within the volume of the bag flow past the acoustophoresis device and carry desired biomolecules out of the bag for collection, while cells and other debris remain within the bag for disposal.

### CROSS-REFERENCE TO RELATED APPLICATIONS

This application claims priority to U.S. Provisional Patent Application Ser. No. 61/950,309, filed on Mar. 10, 2014, the disclosure of which is incorporated herein by reference in its entirety.

### BACKGROUND

The ability to separate a particle/fluid mixture into its separate components is desirable in many applications. Acoustophoresis is the separation of particles using high intensity sound waves, and without the use of membranes or physical size exclusion filters. It has been known that high intensity standing waves of sound can exert forces on particles in a fluid when there is a differential in both density and compressibility, otherwise known as the contrast factor. A standing wave has a pressure profile which appears to “stand” still in time. The pressure profile in a standing wave contains areas of net zero pressure at its nodes and anti-nodes. Depending on the density and compressibility of the particles, they will be trapped at the nodes or anti-nodes of the standing wave. The higher the frequency of the standing wave, the smaller the particles that can be trapped.

Growth in the field of biotechnology has been due to many factors, some of which include the improvements in the equipment available for bioreactors. Improvements in equipment have allowed for larger volumes and lower cost for the production of biologically derived materials such as monoclonal antibodies and recombinant proteins. One of the key components used in the manufacturing processes of new biologically based pharmaceuticals is the bioreactor and the ancillary processes associated therewith.

A modern bioreactor is a very complicated piece of equipment. It provides for, among other parameters, the regulation of fluid flow rates, gas content, temperature, pH and oxygen content. All of these parameters can be tuned to allow the cell culture to be as efficient as possible of producing the desired biomolecules from the bioreactor process. One process for using a bioreactor field is the perfusion process. The perfusion process is distinguished from the fed-batch process by its lower capital cost and higher throughput.

In the fed-batch process, a culture is seeded in a bioreactor. The gradual addition of a fresh volume of selected nutrients during the growth cycle is used to improve productivity and growth. The product, typically a monoclonal antibody or a recombinant protein, is recovered after the culture is harvested. Separating the cells, cell debris and other waste products from the desired product is currently performed using various types of filters for separation, such as diatomaceous earth (DE) filters and membrane filters. Such filters are expensive and become clogged and non-functional as the bioreactor material is processed. A fed-batch bioreactor also has high start-up costs, and generally requires a large volume to obtain a cost-effective amount of product at the end of the growth cycle, and such processes include large amounts of non-productive downtime.

A perfusion bioreactor processes a continuous supply of fresh media that is fed into the bioreactor while growth-inhibiting byproducts are constantly removed. The nonproductive downtime can be reduced or eliminated with a perfusion bioreactor process. The cell densities achieved in perfusion culture (30-100 million cells/mL) are typically higher than for fed-batch modes (5-25 million cells/mL). However, a perfusion bioreactor requires a cell retention device to prevent escape of the culture when byproducts are being removed. These cell retention systems add a level of complexity to the perfusion process, requiring management, control, and maintenance for successful operation. Operational issues such as malfunction or failure of the cell retention equipment has previously been a problem with perfusion bioreactors. This has limited their attractiveness in the past.

It would be desirable to provide means that can reduce the cost and effort of using bioreactors and separating the desired products from the cells that make them.

### BRIEF DESCRIPTION

The present disclosure relates, in various embodiments, to systems for producing biomolecules such as recombinant proteins or monoclonal antibodies, and to processes for separating these desirable products from a cell culture in a disposable bioreactor system. Generally, the bioreactor includes an acoustophoretic device for producing multi-dimensional standing waves, which is located near an outlet port for the bioreactor. The standing waves are used to hold the cell culture and other solids in place within the bioreactor. The liquid medium containing the desired biological products/biomolecules flows out of the bioreactor and is collected. The biomolecules can then be separated/harvested from the liquid medium.

For three-dimensional acoustic fields, Gor′kov's formulation can be used to calculate the acoustic radiation force F_{ac} applicable to any sound field. The primary acoustic radiation force F_{ac} is defined as a function of a field potential U,

F_{A}=−∇(U),

where the field potential U is defined as

$$U = {V}_{0} [ \frac{〈 {p}^{2} 〉}{2 {ρ}_{f} {c}_{f}^{2}} {f}_{1} - \frac{3 {ρ}_{f} 〈 {u}^{2} 〉}{4} {f}_{2} ] ,$$

and f₁ and f₂ are the monopole and dipole contributions defined by

$${f}_{1} = 1 - \frac{1}{{Λσ}^{2}} , {f}_{2} = \frac{2 ( Λ - 1 )}{2 Λ + 1} ,$$

where p is the acoustic pressure, u is the fluid particle velocity, Λ is the ratio of cell density ρₚ to fluid density ρ_{f}, σ is the ratio of cell sound speed cₚ to fluid sound speed c_{f}, Vₒ is the volume of the cell, and < > indicates time averaging over the period of the wave.

Perturbation of the piezoelectric crystal in an ultrasonic transducer in a multimode fashion allows for generation of a multidimensional acoustic standing wave. Generation of a standing wave using a piezoelectric crystal specifically designed to deform in a multimode fashion as it provides a piston-like oscillation to a fluid at designed frequencies, allows for generation of a multi-dimensional acoustic standing wave. The multi-dimensional acoustic standing wave may be generated at distinct modes of the piezoelectric crystal such as the 3×3 mode that would generate multidimensional acoustic standing waves. A multitude of multidimensional acoustic standing waves may also be generated by allowing the piezoelectric crystal to vibrate through many different mode shapes. Thus, the crystal would excite multiple modes such as a 0×0 mode (i.e. a piston mode) to a 1×1, 2×2, 1×3, 3×1, 3×3, and other higher order modes and then cycle back through the lower modes of the crystal (not necessarily in straight order). This switching or dithering of the crystal between modes allows for various multidimensional wave shapes, along with a single piston mode shape to be generated over a designated time.

Disclosed in various embodiments are disposable bioreactor systems, comprising: a bag having a first end, a second end, and a port at the first end; an acoustophoresis device disposed about the first end of the bag, the acoustophoresis device being separable from the bag; and an actuating mechanism operably connected to the second end of the bag, the actuating mechanism being configured to move the second end of the bag towards the acoustophoresis device at the first end of the bag.

The bag may comprise multiple layers of differentially functioning polymers. The bag can be corrugated, thereby allowing the bag to collapse on itself.

The bag may further include a neck portion disposed at the first end of the bag, the neck portion including the port. In such embodiments, the acoustophoresis device can be disposed on the neck portion upstream of the port. The acoustophoresis device can be configured to generate a multi-dimensional standing wave upstream of the port.

The system may further include an impeller disposed within the bag. The side of the bag may include a liquid-tight access point through which an impeller can be inserted and removed from the interior volume of the bag. Alternatively, the side of the bag may include a jack for connecting to an impeller that is permanently sealed within the interior volume of the bag, the jack being used to provide power to the impeller.

In some embodiments, the actuating mechanism includes: a pan member disposed around the exterior of the second end of the bag; and a screw member operably connected to the pan member. Upon rotation of the screw member, the pan member is configured to move the second end of the bag towards the acoustophoresis device, thereby reducing the volume of the bag.

In other embodiments, the actuating mechanism includes: at least one reel disposed at the first end of the bag; and at least one cable operably connected to the second end of the bag and winding about the at least one reel. Upon movement of the at least one cable about the at least one reel, the at least one cable moves the second end of the bag towards the acoustophoresis device, thereby reducing the volume of the bag.

Also disclosed are methods of separated desired biomolecules from a mixture of solid waste and a liquid permeate, comprising: receiving a bag filled with the desired biomolecules, the solid waste, and the liquid permeate; actuating an actuating mechanism operably connected to a second end of the bag; collapsing the bag so that the solid waste and the liquid permeate flow towards a port on a first end of the bag; and generating a multi-dimensional standing wave with an acoustophoresis device disposed upstream of the port, thus reducing the amount of solid waste passing through the port while permitting the biomolecules and the liquid permeate to pass through the port.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates a first embodiment of a bioreactor system in an unactuated condition.

FIG. 2 illustrates the bioreactor system of FIG. 1 in an actuated condition.

FIG. 3 illustrates a second embodiment of a bioreactor system in an unactuated condition.

FIG. 4 illustrates the bioreactor system of FIG. 3 in an actuated condition.

FIG. 5 is a cross-sectional diagram of a conventional ultrasonic transducer.

FIG. 6 is a cross-sectional diagram of an ultrasonic transducer of the present disclosure. An air gap is present within the transducer, and no backing layer or wear plate is present.

FIG. 7 is a cross-sectional diagram of an ultrasonic transducer of the present disclosure. An air gap is present within the transducer, and a backing layer and wear plate are present.

FIG. 8 is a graph of electrical impedance amplitude versus frequency for a square transducer driven at different frequencies.

FIG. 9 illustrates the trapping line configurations for seven of the peak amplitudes of FIG. 8 from the direction orthogonal to fluid flow.

FIG. 10 is a graph showing the relationship of the acoustic radiation force, buoyancy force, and Stokes' drag force to particle size. The horizontal axis is in microns (μm) and the vertical axis is in Newtons (N).

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps, along with any impurities that might result from the manufacture of the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The term “about” can be used to include any numerical value that can vary without changing the basic function of that value. When used with a range, “about” also discloses the range defined by the absolute values of the two endpoints, e.g. “about 2 to about 4” also discloses the range “from 2 to 4.” The term “about” may refer to plus or minus 10% of the indicated number.

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “upwards” and “downwards” are also relative to an absolute reference; an upwards flow is always against the gravity of the earth.

The present application refers to “the same order of magnitude.” Two numbers are of the same order of magnitude if the quotient of the larger number divided by the smaller number is a value less than 10.

The term “agitator” is used herein to refer to any device or system which can be used to cause mixing of a fluid volume, such that material in the fluid volume is dispersed and becomes more homogeneous. The term “impeller” is used to refer to a physical agitator, such as a blade. Examples of agitators which are not impellers may include aerators (which use air).

Bioreactors are useful for making biomolecules such as recombinant proteins or monoclonal antibodies. Very generally, cells are cultured in a bioreactor vessel with media in order to produce the desired product, and the desired product is then harvested by separation from the cells and media. The use of mammalian cell cultures including Chinese hamster ovary (CHO), NSO hybridoma cells, baby hamster kidney (BHK) cells, and human cells has proven to be a very efficacious way of producing/expressing the recombinant proteins and monoclonal antibodies required of today's pharmaceuticals.

Much effort has been put into making disposable bioreactors. Multilayer polymeric bags that are hung in a hollow container have replaced traditional stainless steel fixed volume bioreactor vessels in many applications. Such disposable systems are becoming de rigeur when ramping up the production of biopharmaceutical materials. Conventional physical filter systems, such as diatomaceous earth (DE) and membrane filters, are challenged by the cells, cellular residue, and other debris generated from such fed-batch bioreactors.

In the present disclosure, at least one acoustophoretic device is used in conjunction with a disposable bioreactor bag to facilitate the filtration of the batch within the bioreactor bag itself. For example, at the end of a production cycle for a fed-batch reactor, one or more acoustophoresis devices are connected to a collapsible bioreactor bag at the outflow end of the bioreactor. This enables the containment of the concentrate (i.e. cells, cell debris, and other particulates) within the now-used disposable bioreactor bag while the liquid permeate containing the desired product (e.g. monoclonal antibodies, recombinant proteins) is allowed to flow past the acoustophoresis devices and onto further separation by sterile filtration and/or chromatography equipment.

The acoustophoretic processes of the present disclosure have a major advantage over conventional processes where the disposable bioreactor bag is attached, via hoses, to secondary filtering operations such as a diatomaceous earth depth filter. Conventional processes use extra hoses, pumps and other conveying equipment to bring the bioreactor products to the filtering system. Many times, due to the nature of the cells and cell debris in the bioreactor, the filters in the filtering system need to be changed more than once, even for a relatively small 1000-liter bioreactor. This entails product loss and expense for extra filtration processes.

The acoustophoretic separation technology of the present disclosure employs ultrasonic acoustic standing waves to trap, i.e., hold stationary, particles in a host fluid stream. The particles, CHO cells in this instance, collect at the nodes of the multi-dimensional acoustic standing wave, forming clumps of cells that eventually fall out of the multi-dimensional acoustic standing wave when the clumps have grown to a size large enough to overcome the holding force of the multi-dimensional acoustic standing wave. This is an important distinction from previous approaches where particle trajectories were merely altered by the effect of the acoustic radiation force or were held in place by a piston-mode acoustic standing wave. The scattering of the acoustic field off the particles results in a three dimensional acoustic radiation force, which acts as a three-dimensional trapping field. The acoustic radiation force is proportional to the particle volume (e.g. the cube of the radius) when the particle is small relative to the wavelength. It is proportional to frequency and the acoustic contrast factor. It also scales with acoustic energy (e.g. the square of the acoustic pressure amplitude). For harmonic excitation, the sinusoidal spatial variation of the force is what drives the particles to the stable axial positions within the standing waves. When the acoustic radiation force exerted on the particles is stronger than the combined effect of fluid drag force and buoyancy and gravitational force, the particle is trapped within the acoustic standing wave field. This results in concentration, agglomeration and/or coalescence of the trapped particles. Additionally, secondary inter-particle forces, such as Bjerkness forces, aid in particle agglomeration. These clumps of cells eventually overcome the holding force of the multidimensional acoustic standing wave and, as a result, the clumps of cells can be separated from smaller desirable biomolecules through enhanced gravitational settling of the clumps of cells.

One specific application for the acoustophoresis device is in the processing of bioreactor materials. It is important to be able to filter all of the cells and cell debris from the expressed materials that are in the fluid stream. The expressed materials are composed of biomolecules such as recombinant proteins or monoclonal antibodies, and are the desired product to be recovered. Through the use of acoustophoresis, the separation of the cells and cell debris is very efficient and leads to very little loss of the expressed materials. This is an improvement over current filtration processes (depth filtration, tangential flow filtration, centrifugation), which show limited efficiencies at high cell densities, so that the loss of the expressed materials in the filter beds themselves can be up to 5% or greater of the expressed materials (monoclonal antibodies and recombinant proteins) produced by the cells in the bioreactor. The use of mammalian cell cultures including Chinese hamster ovary (CHO), NSO hybridoma cells, baby hamster kidney (BHK) cells, and human cells has proven to be a very efficacious way of producing/expressing the recombinant proteins and monoclonal antibodies required of today's pharmaceuticals. The filtration of the mammalian cells and the mammalian cell debris through acoustophoresis aids in greatly increasing the yield of the bioreactor.

In this regard, the contrast factor is the difference between the compressibility and density of the particles and the fluid itself. These properties are characteristic of the particles and the fluid themselves. Most cell types present a higher density and lower compressibility than the medium in which they are suspended, so that the acoustic contrast factor between the cells and the medium has a positive value. As a result, the axial acoustic radiation force (ARF) drives the cells, with a positive contrast factor, to the pressure nodal planes, whereas cells or other particles with a negative contrast factor are driven to the pressure anti-nodal planes. The radial or lateral component of the acoustic radiation force helps trap the cells. The radial or lateral component of the ARF is larger than the combined effect of fluid drag force and gravitational force.

As the cells agglomerate at the nodes of the standing wave, there is also a physical scrubbing of the cell culture media that occurs whereby more cells are trapped as they come in contact with the cells that are already held within the standing wave. This generally separates the cells from the cell culture media. The expressed biomolecules remain in the nutrient fluid stream (i.e. cell culture medium).

Desirably, the ultrasonic transducer(s) generate a three-dimensional or multi-dimensional acoustic standing wave in the fluid that exerts a lateral force on the suspended particles to accompany the axial force so as to increase the particle trapping capabilities of the standing wave. Typical results published in literature state that the lateral force is two orders of magnitude smaller than the axial force. In contrast, the technology disclosed in this application provides for a lateral force to be of the same order of magnitude as the axial force.

It is also possible to drive multiple ultrasonic transducers with arbitrary phasing. In other words, the multiple transducers may work to separate materials in a fluid stream while being out of phase with each other. Alternatively, a single ultrasonic transducer that has been divided into an ordered array may also be operated such that some components of the array will be out of phase with other components of the array.

Three-dimensional (3-D) or multi-dimensional acoustic standing waves are generated from one or more piezoelectric transducers, where the transducers are electrically or mechanically excited such that they move in a multi-excitation mode. The types of waves thus generated can be characterized as composite waves, with displacement profiles that are similar to leaky symmetric (also referred to as compressional or extensional) Lamb waves. The waves are leaky because they radiate into the water layer, which result in the generation of the acoustic standing waves in the water layer. Symmetric Lamb waves have displacement profiles that are symmetric with respect to the neutral axis of the piezoelectric element, which causes multiple standing waves to be generated in a 3-D space. Through this manner of wave generation, a higher lateral trapping force is generated than if the piezoelectric transducer is excited in a “piston” mode where only a single, planar standing wave is generated. Thus, with the same input power to a piezoelectric transducer, the 3-D or multi-dimensional acoustic standing waves can have a higher lateral trapping force which may be up to and beyond 10 times stronger than a single acoustic standing wave generated in piston mode.

It may be necessary, at times, due to acoustic streaming, to modulate the frequency or voltage amplitude of the standing wave. This may be done by amplitude modulation and/or by frequency modulation. The duty cycle of the propagation of the standing wave may also be utilized to achieve certain results for trapping of materials. In other words, the acoustic beam may be turned on and shut off at different frequencies to achieve desired results.

The lateral force of the total acoustic radiation force (ARF) generated by the ultrasonic transducers of the present disclosure is significant and is sufficient to overcome the fluid drag force at high linear velocities of 1 cm/s and beyond. This lateral ARF can thus be used to retain solids (e.g. cells and cell debris) within the disposable bioreactor bag while liquid permeate escapes at these relatively fast flow rates. This is true for both fed-batch bioreactors and perfusion bioreactors.

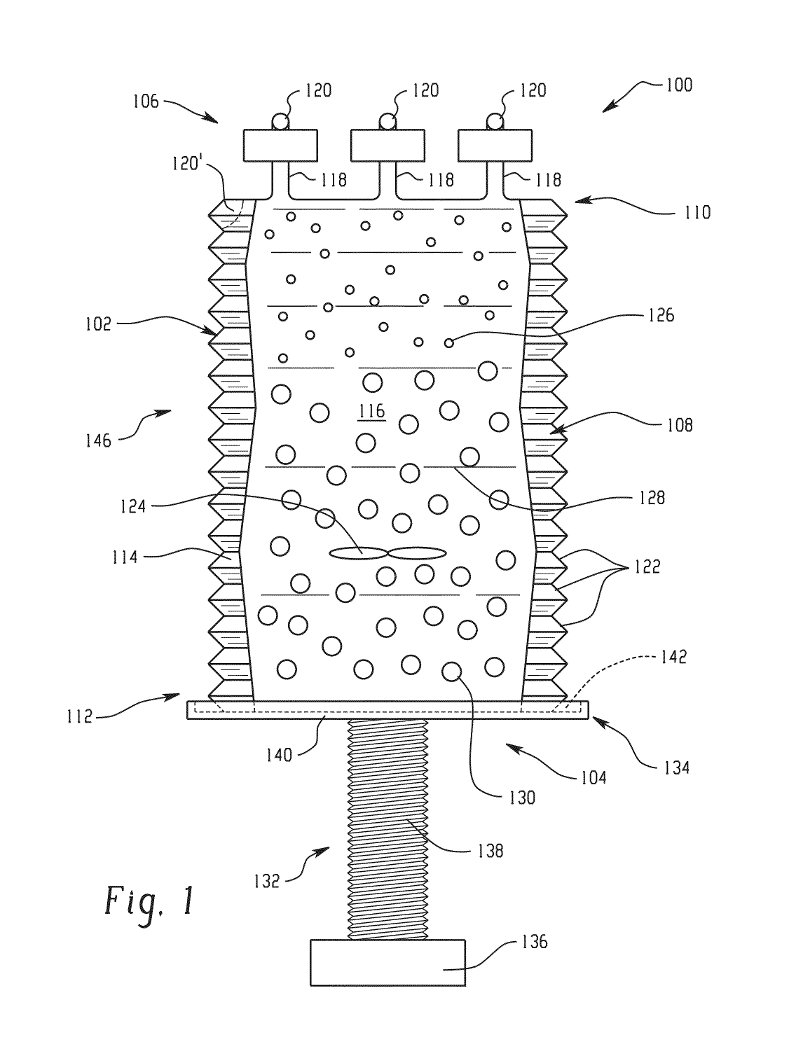

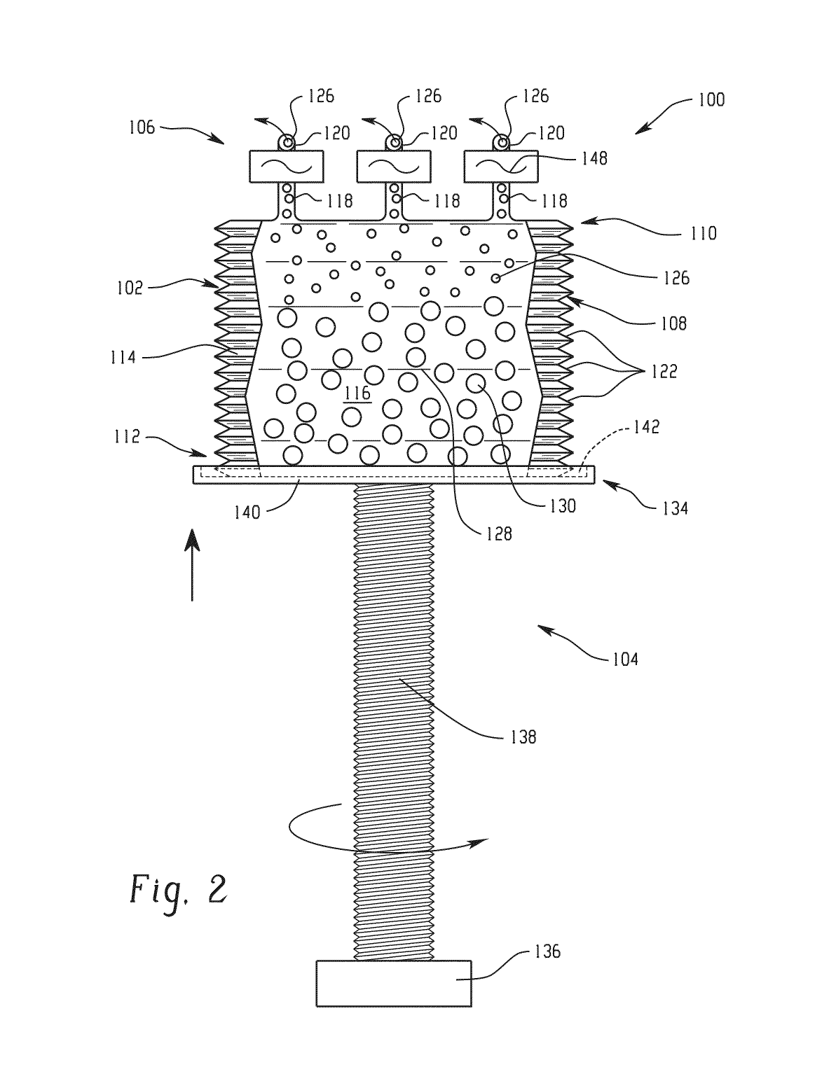

FIGS. 1 and 2 illustrate a first embodiment of a disposable bioreactor system 100 of the present disclosure. As described in more detail below, the system 100 includes a bag 102, an actuating member 104 operably connected to a portion of the bag 102, and at least one acoustophoresis device 106 operably connected to a portion of the bag 102 and spaced from the actuating member 104.

The bag 102 includes a main bag body 108 having a first end 110 and an opposing second end 112. Extending between the first and second ends 110 and 112 is an exterior surface 114 of the main bag body 108. The main bag body 108 also defines a bag interior volume 116 bounded by the exterior surface 114 and extending between the first and second ends 110 and 112. The first end 110 of the main bag body 108 includes one or more ports 120. As illustrated here, the bag is shaped to include one or more neck portions 118, each neck portion 118 including a port 120 at its distal end from the interior volume of the bag for allowing release of biomaterials, as described in more detail below. As shown in FIG. 1, the first end 110 includes three neck portions 118; however, the first end 110 can include any desired number of neck portions 118. In some instances, the starting materials for the bioreactor can be introduced into the bag 102 through the port 120. In other examples, a secondary port 120′ can be provided on the main bag 108 adjacent the first end 110, through which the starting materials can be introduced into the bag 102.

The main bag body 108 is made from at least one polymer layer (e.g., polyethylene, polyurethane, polypropylene, and the like). In other examples, the bag 102 is made from multiple layers of differentially functioning polymer layers. Those polymer layers may function as a waterproof layer, as a layer that provides strength, etc. For example, in some instances, the exterior (i.e. outermost layer) of the bag is a polyethylene terephthalate (PET) polymer. A middle or central layer of the bioreactor bag can be typically ethylene vinyl alcohol (EVOH) or polyvinyl acetate (PVA). The interior layer (contacting the bioreactor cell culture medium) is typically a polyethylene polypropylene such as low-density polyethylene or very low density polyethylene. The bag has a large interior volume, generally of at least one liter, up to 1000 liters, and even larger as desired.

As illustrated here, the bag is corrugated, or in other words contains corrugations 122. These corrugations 122 allow the bag 102 to collapse on itself and fold more easily, reducing the volume of the bag, as described in more detail below.

The acoustophoresis device 106 is disposed at the first end 110 of the main bag body 108. As illustrated here, the acoustophoresis device 106 is disposed on/about the neck portion 118, upstream of the port 120. When more than one neck portions 118 are present, an individual acoustophoresis device 106 can be disposed on each neck portion 118. Thus, multi-dimensional acoustic standing waves can be generated upstream of each port 120, reducing the amount of cells and cell debris that exit from each port with the liquid permeate. It will be appreciated that the acoustophoresis devices 106 are separable (i.e., removable) from the main bag body 108 (i.e., from the first end portion 110).

In some embodiments, an agitator, such as impeller 124, is disposed within the bag 102. The agitator is used to circulate the liquid permeate 128 and cells 130 disposed within the interior volume 116 of the bag. The impeller 124 is depicted as a set of rotating blades, though any type of system that causes circulation is contemplated. The impeller can be inserted into the bag through an access point which includes a water-proof cuff and subsequently removed after the batch process is completed. Alternatively, the impeller can be made of a disposable material (e.g. plastic) which is sealed inside the bag and simply connected to a jack on the side of the bag to provide power. As yet another alternative, the bioreactor can be placed on a rocking support that creates a non-invasive rocking motion, for example in the WAVE™ bioreactor systems offered by GE Healthcare Life Sciences.

The bioreactor system 100 permits growth of a seed culture through a growth/production cycle, during which time debris, cellular waste and cells 130 accumulate in the bag 102 and the desired product (e.g., the biomolecules 126, which can include monoclonal antibodies, recombinant proteins, hormones, etc.) is produced as well. The biomaterials 126 and the liquid permeate 128 are then harvested at the end of the production cycle and collected out of the bag 102, while the cells 130 and other solid waste remain in the bag 102.

To harvest the biomaterials 126 and the liquid permeate 128, the actuating mechanism 104 is operably connected to the second end 112 of the main bag body 108 to move the second end 112 towards the acoustophoresis device 106 (i.e., towards the first end 110 of the bag 102) and reduce the volume of the bag. In one exemplary embodiment, as shown in FIGS. 1 and 2, the actuating mechanism 104 includes a screw member 132 and a pan member 134 operably connected to the screw member 132. As shown, the screw member 132 and the pan member 134 are disposed at the second end 112 of the bag 102, while the acoustophoresis device 106 is disposed at the first end 110 of the bag 102. It will be appreciated that the actuating mechanism 104 and the acoustophoresis device 106 are disposed at opposing ends 110, 112 of the bag 102 from each other.

The screw member 132 includes a head portion 136 and a threaded portion 138. The screw member 132 is connected to the pan member 134. The pan member 134 includes a pan member body 140 that defines a pan member cavity 142. The pan member cavity 142 is sized and dimensioned to fit around the main bag body 108 (i.e., to receive the main body bag 108). A portion of the pan member body 140 is operably connected to the threaded portion 138 of the screw member 132 (e.g., by welding).

The head portion 136 of the screw member 132 is fixed in place relative to the acoustophoretic devices 106. In other words, there is a constant distance between the head portion 136 and the acoustophoretic devices 106. This can be accomplished, for example, using a frame.

As shown in FIG. 2, upon rotation of the screw member 132, the threaded portion 138 thereof moves the pan member body 140 longitudinally (i.e., in an up and down direction). This rotation can be induced, for example, by manual means, by rotating a screwdriver at a distal end of the threaded portion 138, or where the head portion 136 rotates the threaded portion 138 (e.g., an electronic controller).

The pressure applied by the pan member body 140 to the main bag body 108 causes the interior volume 116 to decrease. Stated another way, the second end 112 of the main bag body 104 is collapsed by the pan member 134. Advantageously, the corrugations 122 collapse (i.e., like an accordion) to facilitate the collapse of the bag 102. Consequently, the contents of the bag 102 (e.g., the biomaterials 126, the liquid permeate 128, and the cells 130) are moved by the pan member 134 towards the first end 110 of the bag and towards the acoustophoresis devices 106. Upon passage through the acoustophoresis devices 106, the biomaterials 126 and the liquid permeate 128 exit through the ports 120 and are collected by a collection unit (not shown) within the bioreactor system 100. On the other hand, the cells 130 and other solid materials remain in the bag 102 for disposal.

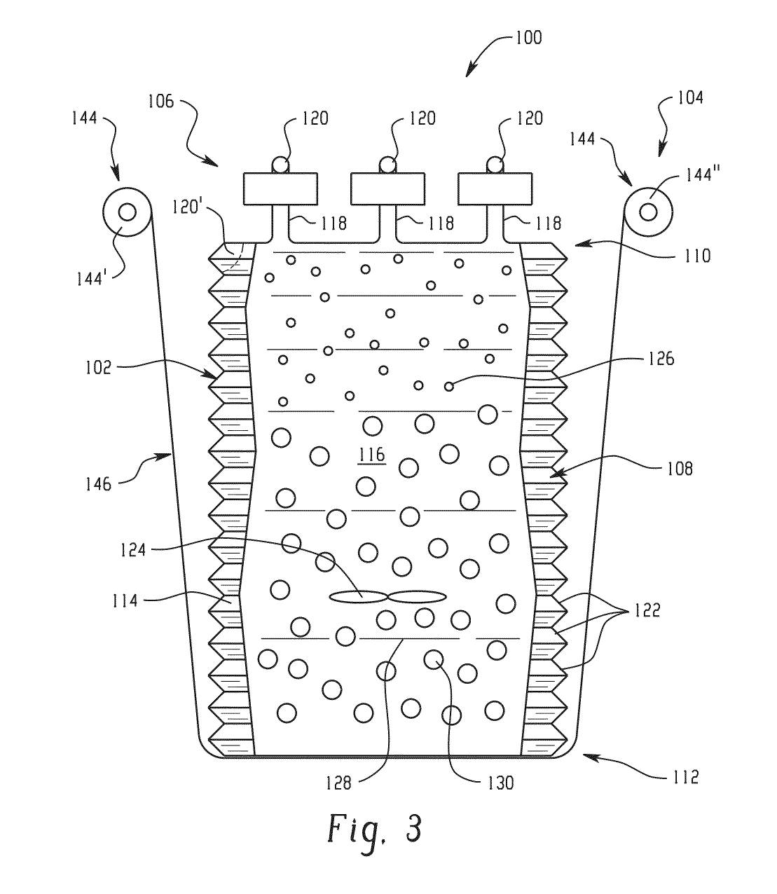

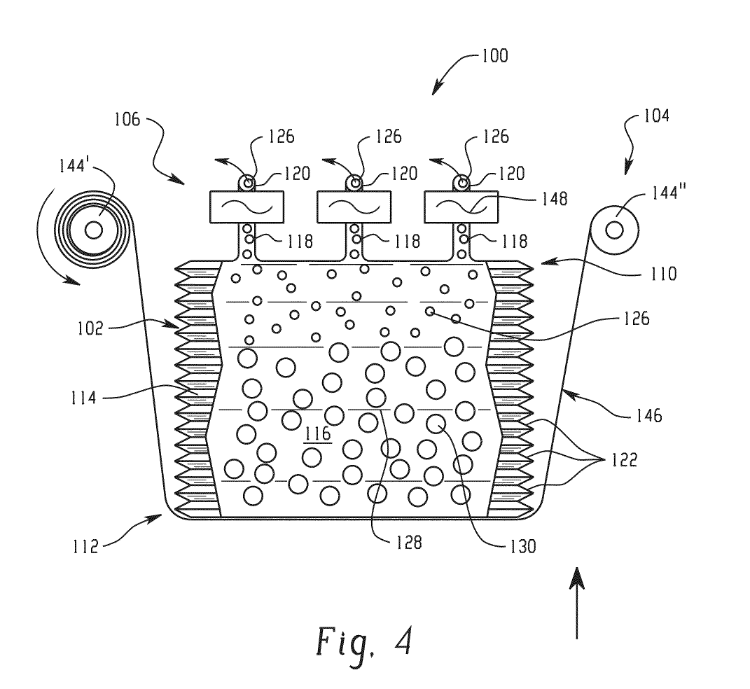

Turning to FIGS. 3 and 4, in another exemplary embodiment, the actuating mechanism 104 includes at least one reel 144 disposed at the first end 110 of the main bag body 104, and at least one cable 146 operably connected to the second end 112 of the main bag body 104. The reels 144 are attached to a portion (i.e., a wall) of the bioreactor system 100. As shown in FIGS. 3 and 4, a single cable 146 is wrapped around a pair of reels 144 disposed on opposing lateral sides of the main bag body 104 (i.e., on “left” and “right” sides of the main bag body 104); although it will be appreciated that any number of cables 146 or reels 144 can be used.

In some embodiments, only one of the reels 144′ is movable, while the other reel 144″ is stationary (i.e. does not rotate and acts as a fixed point). The movable reel 144′ can be rotated by any suitable means (e.g., manually, an electronic controller, and the like). Consequently, when the movable reel 144′ is rotated, the cable 146 wraps around the rotated reel 144′ while the stationary reel 144″ maintains the tension in the cable 146. Upon rotation of the movable reel 144′, slack in the cable 146 is taken up as the cable 146 wraps around the movable reel 144′. The tension in the cable 146 causes the second end 112 of the main bag body 108 to collapse to move the second end 112 towards the acoustophoresis device 106, thereby causing the interior volume 116 of the main bag body 108 to decrease. Advantageously, the corrugations 122 collapse (i.e., like an accordion) to facilitate the collapse of the bag 102. Consequently, the contents of the bag 102 (e.g., the biomaterials 126, the liquid permeate 128, and the cells 130) are moved by the cable 146 towards the first end 110 of the bag 102 and, thus, towards the acoustophoresis device 106 to be separated.

During actuation of the actuating member 104, a multi-dimensional standing wave 148 is generated using the acoustophoresis device 106. The standing wave 148, as described above, has a frequency which allows the biomaterials 126 and the liquid permeate 128 to exit the bag 102 via the ports 120, while trapping the cells 130 so that they do not exit the bag 102 through the ports 120, or at least heavily reducing the number of cells that exit the bag. Once all (or a desired portion) of the biomaterials 126 and the liquid permeate 128 are collected, the bag 102, with the cells 130 still inside, is disconnected from the acoustophoresis device 106 and the bioreactor system 100 and is disposed of.

By the incorporation of an acoustophoresis device or acoustophoresis devices on the outflow ports of the bioreactor at the end of its process cycle, the contents of the bioreactor may be passed through the acoustophoresis filter or filters by collapsing or rolling up the bioreactor bag. The cells, cell debris and other solids are caught in the standing waves, clumped up into larger groups and fall back into the bioreactor bag due to the force of gravity.

The two exemplary embodiments shown in FIGS. 1-4 can be used for both fed-batch processes and perfusion processes. For fed-batch processes, the actuating mechanism 104 is used once at the end of the production cycle. For perfusion processes, the actuating mechanism would be operated to reduce the interior volume of the bag and expel liquid permeate and desired biomolecules past the acoustophoretic devices 106. Then, the actuating mechanism would be operated to increase the interior volume and permit additional medium or cells to be added to the bag.

It is also noted that in the embodiments of FIGS. 1-4, the ports 120 that permit liquid permeate to exit the interior volume are located at an upper end of the bag. This is generally done because the cells 130 are denser than the liquid permeate, and thus will drop downwards under the influence of gravity once they agglomerate. Putting the ports 120 at the bottom end would result in their being clogged. However, it is also contemplated that the ports 120 and neck portions 118 of the bag could be located at the upper end of the bag and then extend laterally to the sides.

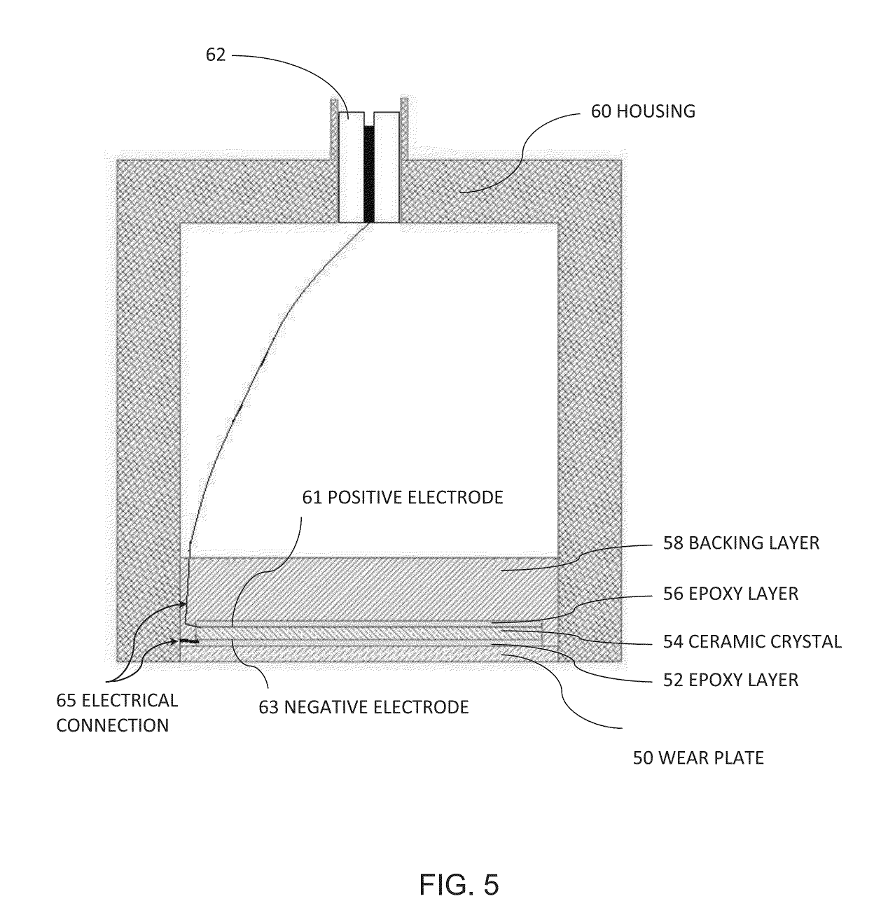

It may be helpful now to describe the ultrasonic transducer(s) used in the acoustophoretic filtering device in more detail. FIG. 5 is a cross-sectional diagram of a conventional ultrasonic transducer. This transducer has a wear plate 50 at a bottom end, epoxy layer 52, ceramic crystal 54 (made of, e.g. PZT), an epoxy layer 56, and a backing layer 58. On either side of the ceramic crystal, there is an electrode: a positive electrode 61 and a negative electrode 63. The epoxy layer 56 attaches backing layer 58 to the crystal 54. The entire assembly is contained in a housing 60 which may be made out of, for example, aluminum. An electrical adapter 62 provides connection for wires to pass through the housing and connect to leads (not shown) which attach to the crystal 54. Typically, backing layers are designed to add damping and to create a broadband transducer with uniform displacement across a wide range of frequency and are designed to suppress excitation at particular vibrational eigen-modes. Wear plates are usually designed as impedance transformers to better match the characteristic impedance of the medium into which the transducer radiates.

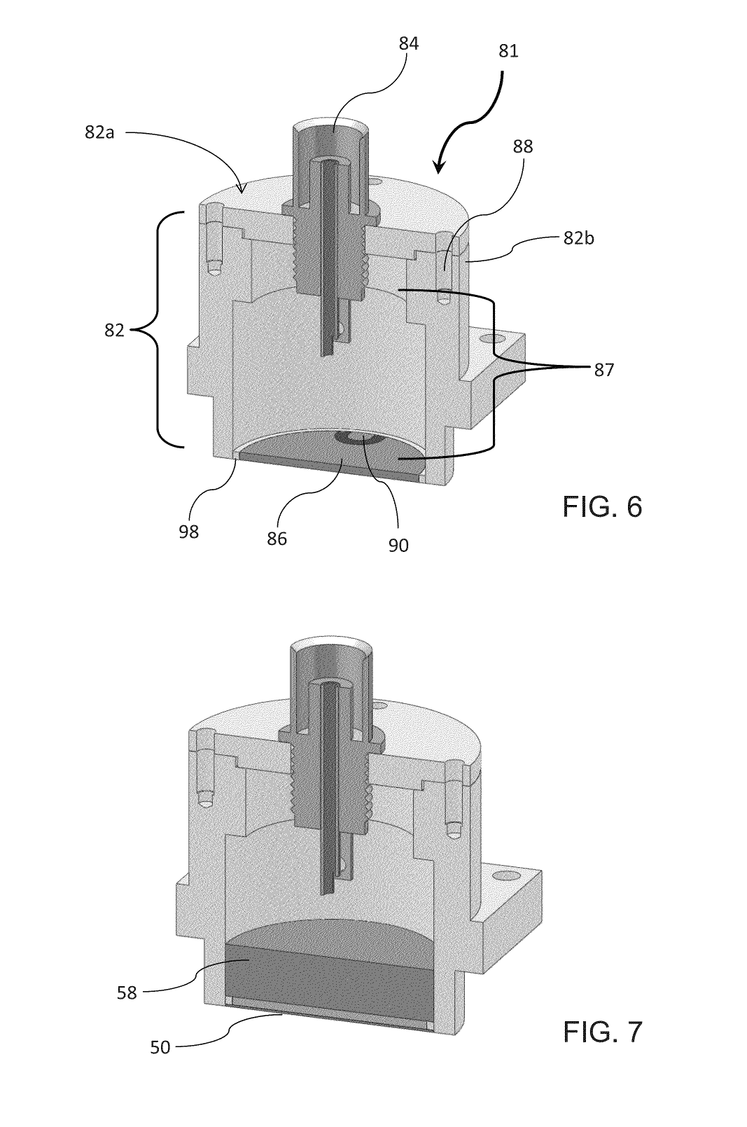

FIG. 6 is a cross-sectional view of an ultrasonic transducer 81 of the present disclosure. Transducer 81 has an aluminum housing 82. A piezoelectric ceramic crystal is utilized to generate the ultrasonic acoustic wave or waves. The piezoelectric crystal is a mass of perovskite ceramic crystals, each consisting of a small, tetravalent metal ion, usually titanium or zirconium, in a lattice of larger, divalent metal ions, usually lead or barium, and O²⁻ ions. As an example, a PZT (lead zirconate titanate) crystal 86 defines the bottom end of the transducer, and is exposed from the exterior of the housing. The crystal is supported on its perimeter by a small elastic layer 98, e.g. silicone or similar material, located between the crystal and the housing. Put another way, no wear layer is present.

Screws (not shown) attach an aluminum top plate 82a of the housing to the body 82b of the housing via threads 88. The top plate includes a connector 84 to pass power to the PZT crystal 86. The bottom and top surfaces of the PZT crystal 86 are each connected to an electrode (positive and negative), such as silver or nickel. A wrap-around electrode tab 90 connects to the bottom electrode and is isolated from the top electrode. Electrical power is provided to the PZT crystal 86 through the electrodes on the crystal, with the wrap-around tab 90 being the ground connection point. Note that the crystal 86 has no backing layer or epoxy layer as is present in FIG. 30. Put another way, there is an air gap 87 in the transducer between aluminum top plate 82a and the crystal 86 (i.e. the air gap is completely empty). A minimal backing 58 and/or wear plate 50 may be provided in some embodiments, as seen in FIG. 7.

The transducer design can affect performance of the system. A typical transducer is a layered structure with the ceramic crystal bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure the transducers, there is no wear plate or backing, allowing the crystal to vibrate in one of its eigenmodes with a high Q-factor. The vibrating ceramic crystal/disk is directly exposed to the fluid flowing through the flow chamber.

Removing the backing (e.g. making the crystal air backed) also permits the ceramic crystal to vibrate at higher order modes of vibration with little damping (e.g. higher order modal displacement). In a transducer having a crystal with a backing, the crystal vibrates with a more uniform displacement, like a piston. Removing the backing allows the crystal to vibrate in a non-uniform displacement mode. The higher order the mode shape of the crystal, the more nodal lines the crystal has. The higher order modal displacement of the crystal creates more trapping lines, although the correlation of trapping line to node is not necessarily one to one, and driving the crystal at a higher frequency will not necessarily produce more trapping lines.

In some embodiments, the crystal may have a backing that minimally affects the Q-factor of the crystal (e.g. less than 5%). The backing may be made of a substantially acoustically transparent material such as balsa wood, foam, or cork which allows the crystal to vibrate in a higher order mode shape and maintains a high Q-factor while still providing some mechanical support for the crystal. The backing layer may be a solid, or may be a lattice having holes through the layer, such that the lattice follows the nodes of the vibrating crystal in a particular higher order vibration mode, providing support at node locations while allowing the rest of the crystal to vibrate freely. The goal of the lattice work or acoustically transparent material is to provide support without lowering the Q-factor of the crystal or interfering with the excitation of a particular mode shape.

Placing the crystal in direct contact with the fluid also contributes to the high Q-factor by avoiding the dampening and energy absorption effects of the epoxy layer and the wear plate. Other embodiments may have wear plates or a wear surface to prevent the PZT, which contains lead, contacting the host fluid. This may be desirable in, for example, biological applications such as separating blood. Such applications might use a wear layer such as chrome, electrolytic nickel, or electroless nickel. Chemical vapor deposition could also be used to apply a layer of poly(p-xylylene) (e.g. Parylene) or other polymer. Organic and biocompatible coatings such as silicone or polyurethane are also usable as a wear surface.

In the present systems, the system is operated at a voltage such that the particles are trapped in the ultrasonic standing wave, i.e., remain in a stationary position. The particles are collected in along well defined trapping lines, separated by half a wavelength. Within each nodal plane, the particles are trapped in the minima of the acoustic radiation potential. The axial component of the acoustic radiation force drives the particles, with a positive contrast factor, to the pressure nodal planes, whereas particles with a negative contrast factor are driven to the pressure anti-nodal planes. The radial or lateral component of the acoustic radiation force is the force that traps the particle. The radial or lateral component of the acoustic radiation force is on the same order of magnitude as the axial component of the acoustic radiation force. As discussed above, the lateral force can be increased by driving the transducer in higher order mode shapes, as opposed to a form of vibration where the crystal effectively moves as a piston having a uniform displacement. The acoustic pressure is proportional to the driving voltage of the transducer. The electrical power is proportional to the square of the voltage.

In some embodiments, the pulsed voltage signal driving the transducer can have a sinusoidal, square, sawtooth, or triangle waveform; and have a frequency of 500 kHz to 10 MHz. The pulsed voltage signal can be driven with pulse width modulation, which produces any desired waveform. The pulsed voltage signal can also have amplitude or frequency modulation start/stop capability to eliminate streaming.

The size, shape, and thickness of the transducer determine the transducer displacement at different frequencies of excitation, which in turn affects separation efficiency. Typically, the transducer is operated at frequencies near the thickness resonance frequency (half wavelength). Gradients in transducer displacement typically result in more places for particles to be trapped. Higher order modal displacements generate three-dimensional acoustic standing waves with strong gradients in the acoustic field in all directions, thereby creating equally strong acoustic radiation forces in all directions, leading to multiple trapping lines, where the number of trapping lines correlate with the particular mode shape of the transducer.

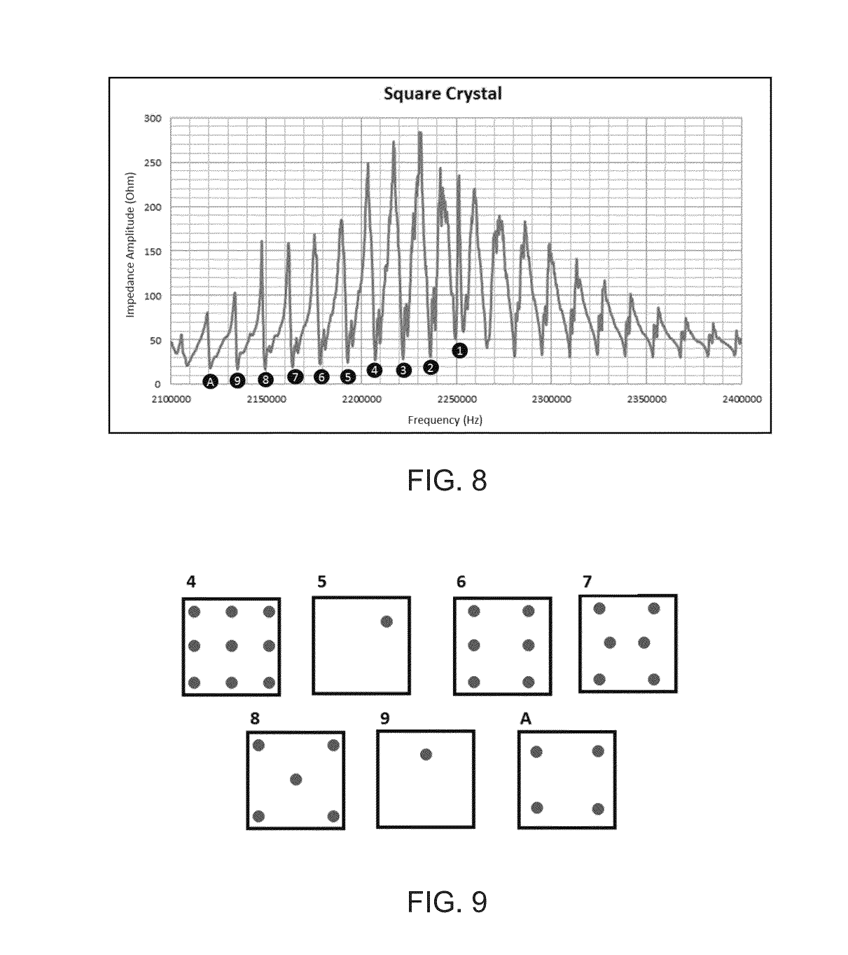

To investigate the effect of the transducer displacement profile on acoustic trapping force and separation efficiencies, an experiment was repeated ten times using a 1″×1″ square transducer, with all conditions identical except for the excitation frequency. Ten consecutive acoustic resonance frequencies, indicated by circled numbers 1-9 and letter A on FIG. 8, were used as excitation frequencies. The conditions were experiment duration of 30 min, a 1000 ppm oil concentration of approximately 5-micron SAE-30 oil droplets, a flow rate of 500 ml/min, and an applied power of 20 W. Oil droplets were used because oil is denser than water, and can be separated from water using acoustophoresis.

FIG. 8 shows the measured electrical impedance amplitude of the transducer as a function of frequency in the vicinity of the 2.2 MHz transducer resonance when operated in a water column containing oil droplets. The minima in the transducer electrical impedance correspond to acoustic resonances of the water column and represent potential frequencies for operation. Numerical modeling has indicated that the transducer displacement profile varies significantly at these acoustic resonance frequencies, and thereby directly affects the acoustic standing wave and resulting trapping force. Since the transducer operates near its thickness resonance, the displacements of the electrode surfaces are essentially out of phase. The typical displacement of the transducer electrodes is not uniform and varies depending on frequency of excitation. As an example, at one frequency of excitation with a single line of trapped oil droplets, the displacement has a single maximum in the middle of the electrode and minima near the transducer edges. At another excitation frequency, the transducer profile has multiple maxima leading to multiple trapped lines of oil droplets. Higher order transducer displacement patterns result in higher trapping forces and multiple stable trapping lines for the captured oil droplets.

As the oil-water emulsion passed by the transducer, the trapping lines of oil droplets were observed and characterized. The characterization involved the observation and pattern of the number of trapping lines across the fluid channel, as shown in FIG. 9, for seven of the ten resonance frequencies identified in FIG. 8. Different displacement profiles of the transducer can produce different (more) trapping lines in the standing waves, with more gradients in displacement profile generally creating higher trapping forces and more trapping lines.

The transducer(s) is/are used to create a pressure field that generates forces of the same order of magnitude both orthogonal to the standing wave direction and in the standing wave direction. When the forces are roughly the same order of magnitude, particles of size 0.1 microns to 300 microns will be moved more effectively towards regions of agglomeration (“trapping lines”). Because of the equally large gradients in the orthogonal acoustophoretic force component, there are “hot spots” or particle collection regions that are not located in the regular locations in the standing wave direction between the transducer and the reflector. Hot spots are located in the maxima or minima of acoustic radiation potential. Such hot spots represent particle collection locations which allow for better wave transmission between the transducer and the reflector during collection and stronger inter-particle forces, leading to faster and better particle agglomeration.

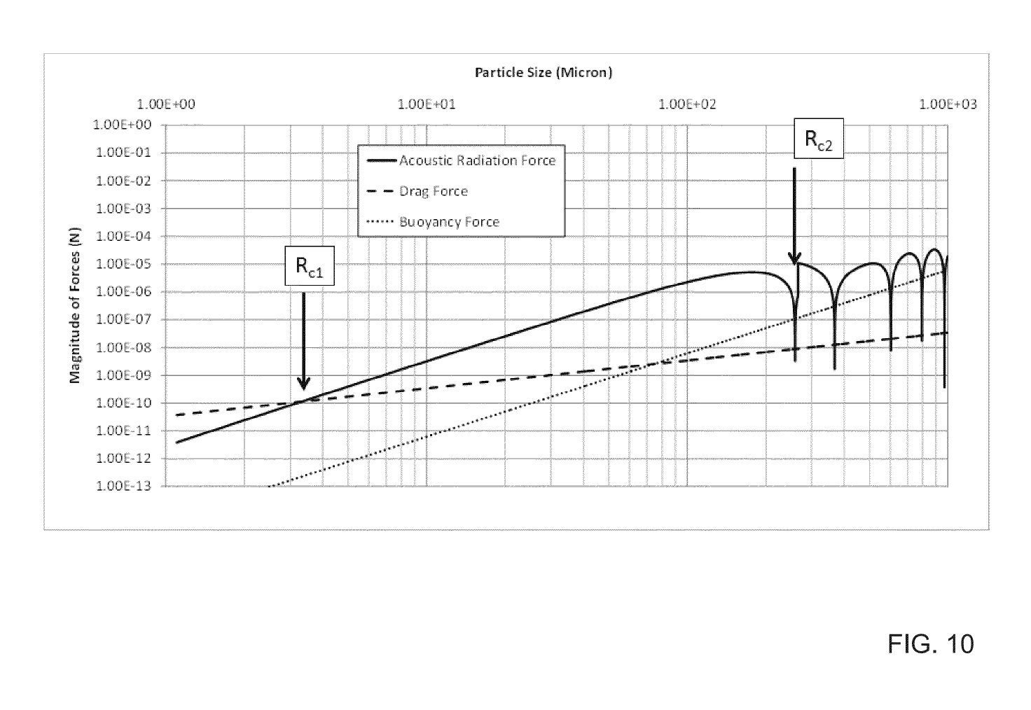

Finally, FIG. 10 is a lin-log graph (linear y-axis, logarithmic x-axis) that shows the scaling of the acoustic radiation force, fluid drag force, and buoyancy force with particle radius. Calculations are done for a typical SAE-30 oil droplet used in experiments. The buoyancy force is a particle volume dependent force, and is therefore negligible for particle sizes on the order of micron, but grows, and becomes significant for particle sizes on the order of hundreds of microns. The fluid drag force scales linearly with fluid velocity, and therefore typically exceeds the buoyancy force for micron sized particles, but is negligible for larger sized particles on the order of hundreds of microns. The acoustic radiation force scaling acts differently. When the particle size is small, the acoustic trapping force scales with the volume of the particle. Eventually, when the particle size grows, the acoustic radiation force no longer increases with the cube of the particle radius, and will rapidly vanish at a certain critical particle size. For further increases of particle size, the radiation force increases again in magnitude but with opposite phase (not shown in the graph). This pattern repeats for increasing particle sizes.

Initially, when a suspension is flowing through the system with primarily small micron sized particles, it is necessary for the acoustic radiation force to balance the combined effect of fluid drag force and buoyancy force for a particle to be trapped in the standing wave. In FIG. 10 this happens for a particle size of about 3.5 micron, labeled as R_{c1}. The graph then indicates that all larger particles will be trapped as well. Therefore, when small particles are trapped in the standing wave, particles coalescence/clumping/aggregation/agglomeration takes place, resulting in continuous growth of effective particle size. As the particle size grows, the acoustic radiation force reflects off the particle, such that large particles will cause the acoustic radiation force to decrease. Particle size growth continues until the buoyancy force becomes dominant, which is indicated by a second critical particle size, R_{c2}, at which size the particles will rise or sink, depending on their relative density with respect to the host fluid. As the particles rise or sink, they no longer reflect the acoustic radiation force, so that the acoustic radiation force then increases. Not all particles will drop out, and those remaining particles will continue to grow in size as well. This phenomenon explains the quick drops and rises in the acoustic radiation force beyond size R_{c2}. Thus, FIG. 10 explains how small particles can be trapped continuously in a standing wave, grow into larger particles or clumps, and then eventually will rise or settle out because of increased buoyancy force.

In biological applications, it is contemplated that all of the parts of the system (e.g. the reaction vessel, tubing leading to and from the bioreactor, the temperature-regulating jacket, etc.) can be separated from each other and be disposable. Avoiding centrifuges and filters allows better separation of the CHO cells without lowering the viability of the cells. The frequency of the transducers may also be varied to obtain optimal effectiveness for a given power.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A disposable bioreactor system, comprising:
a bag having a first end, a second end, and a port at the first end;
at least one acoustophoresis device disposed about the first end of the bag, the at least one acoustophoresis device being separable from the bag; and
an actuating mechanism operably connected to the second end of the bag, the actuating mechanism being configured to move the second end of the bag towards the at least one acoustophoresis device at the first end of the bag.

2. The system of claim 1, wherein the bag comprises multiple layers of differentially functioning polymers.

3. The system of claim 2, wherein the bag is corrugated, thereby allowing the bag to collapse on itself.

4. The system of claim 1, wherein the bag further includes a neck portion disposed at the first end of the bag, the neck portion including the port.

5. The system of claim 4, wherein the at least one acoustophoresis device is disposed on the neck portion upstream of the port.

6. The system of claim 1, wherein the at least one acoustophoresis device is configured to generate a multi-dimensional acoustic standing wave upstream of the port.

7. The system of claim 1, further including an agitator or an impeller disposed within the bag.

8. The system of claim 1, wherein the actuating mechanism includes:
a pan member disposed around the exterior of the second end of the bag; and
a screw member operably connected to the pan member.

9. The system of claim 8, wherein, upon rotation of the screw member, the pan member is configured to move the second end of the bag towards the at least one acoustophoresis device, thereby reducing the volume of the bag.

10. The system of claim 1, wherein the actuating mechanism includes:
at least one reel disposed at the first end of the bag; and
at least one cable operably connected to the second end of the bag and winding about the at least one reel.

11. The system of claim 10, wherein, upon movement of the at least one cable about the at least one reel, the at least one cable moves the second end of the bag towards the at least one acoustophoresis device, thereby reducing the volume of the bag.

12. A method of separated desired biomolecules from a mixture of solid waste and a liquid permeate, the method comprising:
receiving a bag filled with the desired biomolecules, the solid waste, and the liquid permeate;
actuating an actuating mechanism operably connected to a second end of the bag;
collapsing the bag so that the solid waste and the liquid permeate flow towards a port or ports on a first end of the bag; and
generating a multi-dimensional acoustic standing wave with at least one acoustophoresis device disposed upstream of the port or ports, thus reducing the amount of solid waste passing through the port or ports while permitting the biomolecules and the liquid permeate to pass through the port or ports.

13. The method of claim 12, wherein the actuating mechanism includes a pan member disposed around the exterior of the second end of the bag and a screw member operably connected to the pan member, the method further including rotating the screw member to move the pan member towards the at least one acoustophoresis device, thereby reducing the volume of the bag.

14. The method of claim 12, wherein the actuating mechanism includes at least one reel disposed at the first end of the bag and at least one cable operably connected to the second end of the bag and winding about the at least one reel, the method further including moving the at least one cable about the at least one reel to move the second end of the bag towards the at least one acoustophoresis device, thereby reducing the volume of the bag.

15. The method of claim 12, wherein the bag is formed from multiple layers of differentially functioning polymers.

16. The method of claim 15, wherein the bag is corrugated.

17. The method of claim 12, further including disposing of the bag after the bioreactor materials pass through the at least one port, the bag including the solid waste.

18. The method of claim 12, where the multi-dimensional acoustic standing wave is generated by shifting between various excitation modes of a piezoelectric crystal in the at least one acoustophoretic device.

19. The method of claim 12, where the multi-dimensional acoustic standing wave is generated by vibrating a piezoelectric crystal in the at least one acoustophoretic device from a 1×1 mode to higher order modes and back to the 1×1 mode over a fixed period of time.
